(19) United States
(12) Patent Application Publication
(10) Pub. No.: US 2006/0135912 A1
(43) Pub. Date: Jun. 22, 2006

(54) Biodegradable pericardia constraint system and method

(75) Inventors: Ary Chernomorsky, Walnut Creek, CA (US); Mark Gelfand, New York, NY (US); Howard R. Levin, Teaneck, NJ (US)

Correspondence Address: NIXON & VANDERHYE, PC, 901 NORTH GLEBE ROAD, 11TH FLOOR, ARLINGTON, VA, 22203, US

(73) Assignee: G&L Consulting, LLC, New York, NY (US)

(21) Appl. No.: 11/282,694

(22) Filed: Nov. 21, 2005

Related U.S. Application Data
(63) Continuation-in-part of application No. 10/808,397, filed on Mar. 25, 2004.
(60) Provisional application No. 60/628,923, filed on Nov. 19, 2004.
(60) Provisional application No. 60/457,246, filed on Mar. 26, 2003.

(51) Int. Cl. A61M 1/00

(52) U.S. Cl. 604/118

(57) ABSTRACT

A system has been developed for injecting a biodegradable pericardial constraint including: a biodegradable viscoelastic substance (BES); an external injection container for the BES; a cannula having a distal section adapted to be inserted into a pericardial sac of a mammalian heart and a proximal section connectable to the external injection container; wherein BES from the injection container is injected into the pericardial sac through the cannula.

### RELATED APPLICATIONS

This application is a continuation-in-part (CIP) application of U.S. patent application Ser. No. 10/808,397, entitled “Method and System To Treat and Prevent Myocardial Infarct Expansion” filed Mar. 25, 2004, which claims priority under 35 U.S.C. §119(e) to U.S. Provisional application 60/457,246, filed Mar. 26, 2003, and this application also claims priority under 35 U.S.C. §119(e) to U.S. Provisional Patent Application Ser. No. 60/628,923, entitled “Biodegradable Pericardial Constraint”, filed Nov. 19, 2004, the entirety of all of these related applications are incorporated by reference herein.

### BACKGROUND OF INVENTION

A Myocardial Infarction (MI) or heart attack, occurs when the blood supply to some part of the heart muscle (myocardium) is abruptly stopped. This is often due to clotting in a coronary blood vessel. Blood supplying the heart muscle comes entirely from two coronary arteries, both lying along the outside surface of the heart. If one of these arteries or any part of one suddenly becomes blocked, the area of the heart being supplied by the artery dies. The death of a portion of the heart muscle is a myocardial infarct, and the amount of the heart affected by the sudden occlusion will determine the severity of the attack. If the heart continues to function, the dead portion is eventually walled off as new vascular tissue supplies the needed blood to adjacent areas.

According to the American Heart Association, in the year 2000 approximately 1,100,000 new myocardial infarctions occurred in the United States. For 650,000 patients this was their first myocardial infarction, while for the other 450,000 patients this was a recurrent event. Two hundred-twenty thousand people suffering MI die before reaching the hospital. Within one year of the myocardial infarction, 25% of men and 38% of women die. Within 6 years, 22% of Men and 46% of women develop chronic heart failure, of which 67% are disabled.

An MI starts when a coronary artery suddenly becomes occluded and can no longer supply blood to the myocardial tissue. When a myocardial infarction occurs, the myocardial tissue that is no longer receiving adequate blood flow dies and is replaced with scar tissue. Within seconds of a myocardial infarction, the under-perfused myocardial cells no longer contract, leading to abnormal ventricular wall motion, high wall stresses within and surrounding the infarct, and depressed ventricular function. The infarct expansion and ventricular remodeling are caused by these high stresses at the junction between the infracted (not contracting) tissue and the normal myocardium. These high stresses eventually kill or severely depress function in the still viable myocardial cells. This results in a wave of dysfunctional tissue spreading out from the original myocardial infarct region.

Left ventricular remodeling is defined as changes in shape and size of the Left Ventricle (LV) that can follow a MI. The process of LV enlargement can be influenced by three independent factors that is, infarct size, infarct healing and LV wall stress. The process is a continuum, beginning in the acute period and continuing through and beyond the late convalescent period. During the early period after MI the infarcted region is particularly vulnerable to distorting forces. This period of remodeling is called infarct expansion. The infarct expansion phase of remodeling starts on the first day of MI (likely several hours after the beginning of the MI) and lasts up to 14 days. Once healed, the infarcted tissue or “scar” itself is relatively non distensible and much more resistant to further deformation. Therefore late enlargement is due to complex alterations in LV architecture involving both infarcted and non-infarcted zones. This late chamber enlargement is associated with lengthening of the contractile regions rather than progressive infarct expansion. Post infarction LV aneurysm (a bulging out of the thin weak ventricular wall) represents an extreme example of adverse remodeling that leads to progressive deterioration of function with symptoms and signs of congestive heart failure.

Effective treatments for MI are acute and can be only implemented immediately after the occlusion of the coronary vessel. The newest approaches include aggressive efforts to restore patency to occluded vessels broadly called reperfusion therapies. This is accomplished through thrombolytic therapy (with drugs that dissolve the thrombus) or increasingly with primary angioplasty and stents. Reopening the occluded artery within hours of the initial occlusion can decrease tissue death, and thereby decrease the total magnitude of infarct expansion, extension, and ventricular remodeling. These treatments are effective but clearly not satisfactory alone. In many cases, patients arrive at the appropriately equipped hospital too late for these acute therapies. In other cases, their best efforts fail to reopen blood vessels sufficiently to arrest expansion of the infarct. These therapies are also associated with considerable risk to the patient and high cost.

Scientific studies show that constraining the heart in the hours and days following the acute MI can reduce the extent of damage to the heart. Benefits exhibited by constraining the heart during and after the infarct expansion can be traced down to the relationship between the changing geometry of the heart and the stress in the heart muscle that forms the ventricular wall. An example of a treatment for constraining the heart is disclosed in U.S. Patent Application Publication 2004/0193138 A1.

### SUMMARY OF THE INVENTION

A treatment device and method has been invented for clinical use that constrains the heart by placing biodegradable viscoelastic substance acting as a hydraulic heart constrainer in the pericardial sac for a controlled period of time.

An embodiment of a novel treatment device for a biodegradable pericardial constraint comprises: (i) a cannula placed in the pericardial sac, (ii) an external system for delivery of a hydraulic heart constrainer in controlled manner, and (iii) a biodegradable viscoelastic substance (BES) acting as a hydraulic heart constrainer. The treatment method may include the following steps: (i) placement and securing of the cannula for the injection of the biodegradable viscoelastic substance into the pericardial space, (ii) connection of the delivery system containing biodegradable viscoelastic substance to the cannula, (iii) controlled biodegradable viscoelastic substance injection into the pericardial space, (iv) extraction of the cannula, and (v) sealing of the transpericardial incision.

A system has been developed for injecting a biodegradable pericardial constraint comprising: a biodegradable viscoelastic substance (BES); an external injection container for the BES; a cannula having a distal section adapted to be inserted into a pericardial sac of a mammalian heart and a proximal section connectable to the external injection container; wherein BES from the injection container is injected into the pericardial sac through the cannula. The BES may comprises a natural biopolymer, such as lipids, collagen, polysaccharides and polyglyconates, cellulose, gelatin, starch, cross linked collagen gel, a Hyaluronic Acid or a synthetic polymer such as polylactide (PLA), polyglycolide (PGA), poly(lactide-co-glycolide) (PLGA), polyanhydride, PEG and polyorthoesters.

A method has been developed comprising: placement and securing of a cannula to inject a biodegradable viscoelastic substance into a pericardial space of a heart of a mammalian patient; connecting a delivery system containing the biodegradable viscoelastic substance to the cannula, and controlling the injection of the biodegradable viscoelastic substance injection into the pericardial space. The cannula may be inserted through a transpericardial incision in the pericardial sac and the method may include sealing the pericardial sac after extracting the cannula.

A treatment system has been developed for a biodegradable pericardial constraint comprising: a cannula placed in a pericardial sac of a mammalian patient; an external system connectable to the cannula for delivery of a hydraulic heart constrainer in a controlled manner, and a biodegradable viscoelastic substance (BES) to be delivered by the external system to the pericardial sac, wherein the BES acts as a hydraulic heart constrainer when injected into the pericardial sac.

A method has been developed to constrain a mammalian heart comprising: positioning a cannula in a pericardial sac of the heart; introducing a biodegradable viscoelastic substance (BES) though the cannula into the pericardial sac, and extracting the cannula from the pericardial sac after introducing the BES.

### SUMMARY OF THE DRAWINGS

A preferred embodiment and best mode of the invention is illustrated in the attached drawings that are described as follows:

FIGS. 1A, 1B, 1C, and 1D illustrate an initial phase of the treatment procedure of a patient using minimally invasive insertion of the cannula through the subxiphoidal incision into pericardial space. FIG. 1A shows a chest of a person and the internal heart region. FIG. 1B is a line drawing of the chest with the cannula inserted into the heart region. FIG. 1D shows the heart in partial cross-section. FIG. 1C is a cross-sectional diagram of a portion of the heart.

FIG. 2A, 2B illustrate the cannula for injection of a biodegradable viscoelastic substance into the pericardial sac of the heart.

FIG. 2C illustrates the system for delivery of a biodegradable viscoelastic substance into the cannula coupled with anchoring and sealing mechanisms.

FIGS. 3 A, B, C illustrate in cross-section a portion of a heart to show the details of placement and securing of the cannula and injection of the BES into pericardial sac. FIGS. 3 D, E, F illustrate in cross-section a portion of a heart to show the details of extraction of the cannula and sealing of the puncture in the pericardium.

FIGS. 4 A, B illustrate in partial cross-section the extraction of the cannula to show in detail the of closure and sealing of the tissue channel in the pericardium

FIGS. 5 A, B are cross-sectional diagrams of a portion of the heart that illustrate the sealed tissue channel

FIG. 6 A is a cross-section diagram that illustrates the cannula anchoring and the puncture sealing dual action balloon with single lumen.

FIG. 6 B is a cross-section diagram that illustrates an alternative embodiment for the dual action balloon of the cannula.

### DETAILED DESCRIPTION OF THE INVENTION

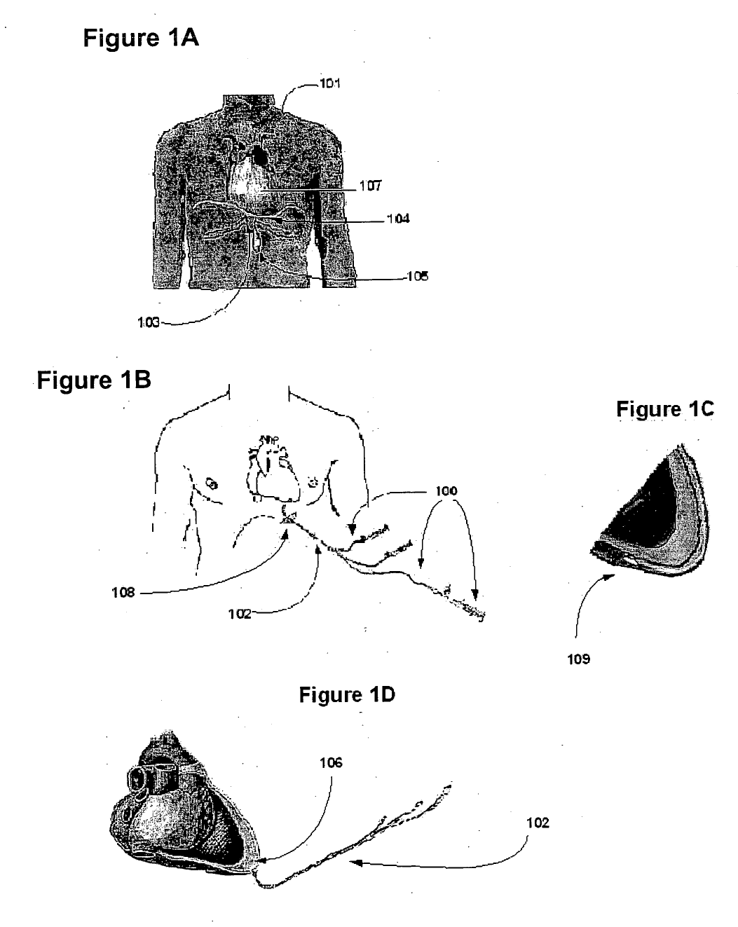

FIG. 1A, B, C, D illustrates the treatment of a patient 101 with the system 100 for injection of BES into the pericardial sac of the heart. The distal end of injection cannula 102 is partially inserted into the pericardial sac of the heart 107. Cannula 102 crosses the patient's skin in the xiphoid area 103 via subxiphoidal incision 105. The diaphragm 104 is incised during surgery down to the pericardial surface. Through this incision 108, the pericardium 107 may be easily visualized and a small incision or a puncture 109 is made in pericardium to accommodate a cannula insertion. The distal tip of the cannula 102 has an opening and is in fluid communication with the pericardial (also called intrapericardial) space 106. The proximal end of the cannula 102 is connected to the delivery system 100 containing the biodegradable viscoelastic substance such as sterile cross linked collagen gel, balloon inflation media such as saline, and tissue sealant such as BioGlu.

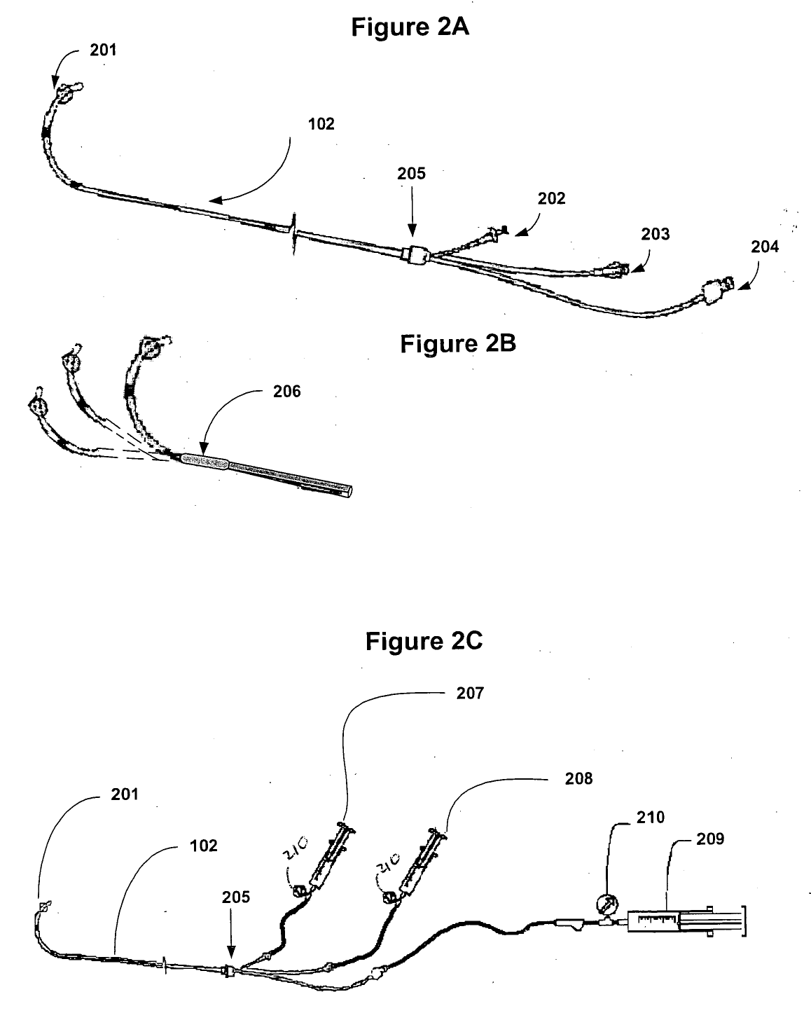

FIGS. 2 A, B illustrate the design of the injection cannula in greater detail. Cannula 102 can be made from 304/306 stainless steel tubing, or memory shape alloy, such as NiTi, or a polymer such as PVC tubing. The cannula has a curvature to accommodate a particular anatomy. Alternatively cannula has a hinge such as metal bellow 206 to adjust the angle of insertion to accommodate a particular anatomy. The cannula also can be a flexible tube capable of assuming various angles to accommodate a particular anatomy.

FIG. 2-A depicts a perspective view of a cannula with an anchoring balloon 201 at the distal end of the cannula and injection lines hub 205 located at the proximal end. Injection line 202 is a tube with a luer lock communicating with balloon inflation media such as saline. Injection line 203 is a tube with a luer lock communicating with balloon inflation media such as tissue sealant. Injection line 204 is a tube with a luer lock communicating with biodegradable viscoelastic substance delivery apparatus 209.

FIG. 2C is a perspective view of the cannula and show the delivery system. The cannula 102 with anchoring/sealing low profile balloon 201 communicates with sources of saline and tissue sealant and the delivery apparatus 209. Delivery apparatus 209 consists of a biodegradable viscoelastic substance containing reservoir, power injector to deliver viscous substances, and pressure gauge 210 to monitor interpericardial pressure during delivery. A majority of the suitable biodegradable viscoelastic substances have a viscosity range of 10000 CST to 15000 CST. To achieve therapeutic effect, the desirable induced interpericardial pressure should be in the range of 12 mmHg to 32 mmHg. The preferred volume range for the biodegradable viscoelastic substance is between 40 ml and 80 ml. To conduct the delivery of such amounts of highly viscous fluids in a controlled manner a power injecting device 209 equipped with pressure gauge 210 such as Breeze inflation pump manufactured by Schneider/Namic Company can be used. Alternatively a custom made power injector can be constructed to accommodate the ergonomics of the procedure Anchoring and sealing balloon 201 of the cannula 102 communicates with a source of saline 207 and tissue sealant 208 via two inflation lumen positioned coaxially or essentially in respect with the shaft of the cannula. Inflation lumens are connected via two way stopcock to reservoir such as a 5 ml B-D syringe containing inflation media such as saline or BioGlue.

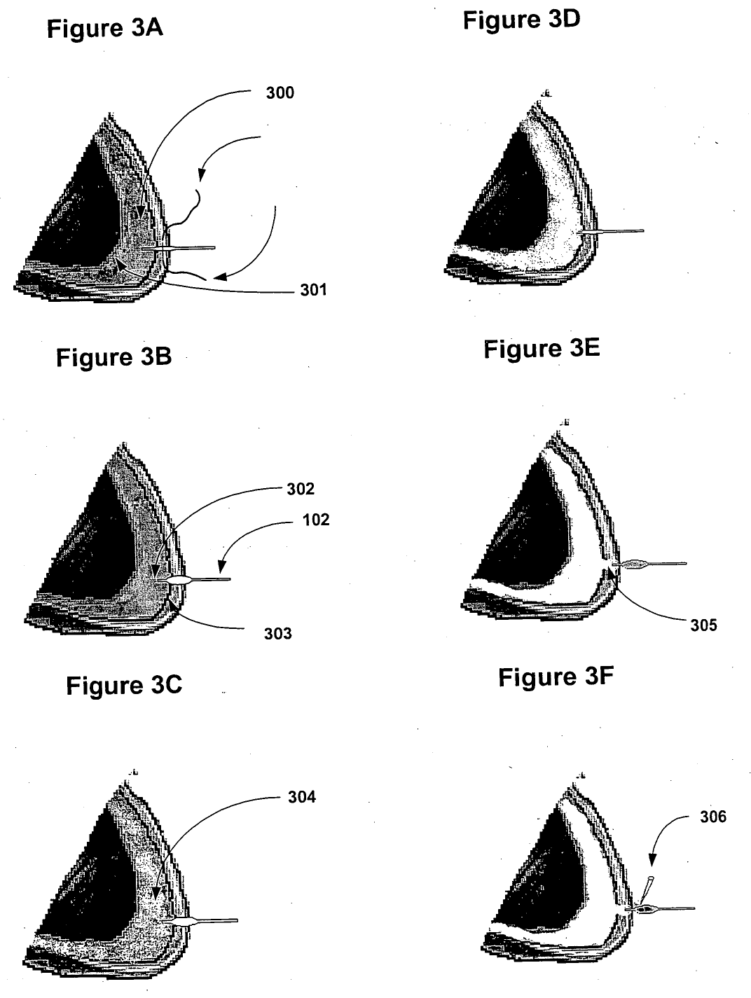

FIGS. 3 A, B, C illustrates method of placement of the hydraulic heart constrainer in greater detail. The cannula 102 is shown inserted into the pericardial space 300 of the heart. Distal tip of the catheter resides in the space between the inner surface of the pericardial sac 303 and the external surface of the heart 301 defined as called pericardial space or intrapericardial space 300. Proximal end of the catheter 102 (not shown) is connected to the delivery system outside of the patient's body. The cannula can be inserted into the pericardium sac using the common clinical technique of pericardiocentesis.

First the pericardium is tapped with a needle. After the position of the needle is confirmed, the needle is withdrawn and replaced with a soft, pigtail catheter using the Seldinger technique. After dilating the needle track, the cannula is advanced over the guidewire into the pericardial space. In order to prevent undesirable oozing of the implantable substance around the cannula during the injection different methods of securing the cannula can be utilized.

A straightforward surgical approach is to place sutures 307 around the cannula in the purse string manner and tighten it up. Another method of securing the cannula in place utilizes inflatable balloon positioned at the distal end of the cannula as shown in FIG. 3B. Once the cannula is placed in desirable position, the balloon is inflated to plug the insertion tunnel and therefore secure the cannula in place and prevent potential oozing of the implantable substance 304 out of the heart and during the injection as shown in FIG. 3C. Yet another method of securing the cannula in place utilizes combination of suturing and purse stringing with the inflatable balloon The inflatable anchoring balloon can be inflated by infusion of saline and utilized for the securing of the cannula only.

The balloon can also be inflated by infusion of the tissue sealant such as BioGlue produced by CryoLife Inc. It will enable inflatable balloon to provide a dual function of anchoring the cannula in place and sealing of the transpericardial incision at the end of the procedure.

FIGS. 3 D, C, F illustrate the final phases of the procedure including cannula extraction and transpericardial puncture sealing. At the end of the clinical procedure when infusion of the BES is completed the anchoring balloon is partially deflated as shown in FIG. 3D, and the cannula is pulled out of the tissue channel 305 created by transpericardial puncture as shown in FIG. 3E. Once the distal surface of the balloon is out of the incision, balloon is immediately reinflated with tissue sealant and firmly pressed against the incision as shown in FIG. 3E, thus creating a plug preventing injected substance 304 from leaking out. After this maneuver, the tip of the inflated balloon is perforated in a controlled manner and the contained sealant is released as shown FIG. 3F. A small gauge needle 306 can be utilized to perforate the balloon by inducing a few orifices preferably circumferentially around the balloon. The released sealant is deposited in intimate proximity of the wound edges and over the opening of the wound sealing the tissue channel 305.

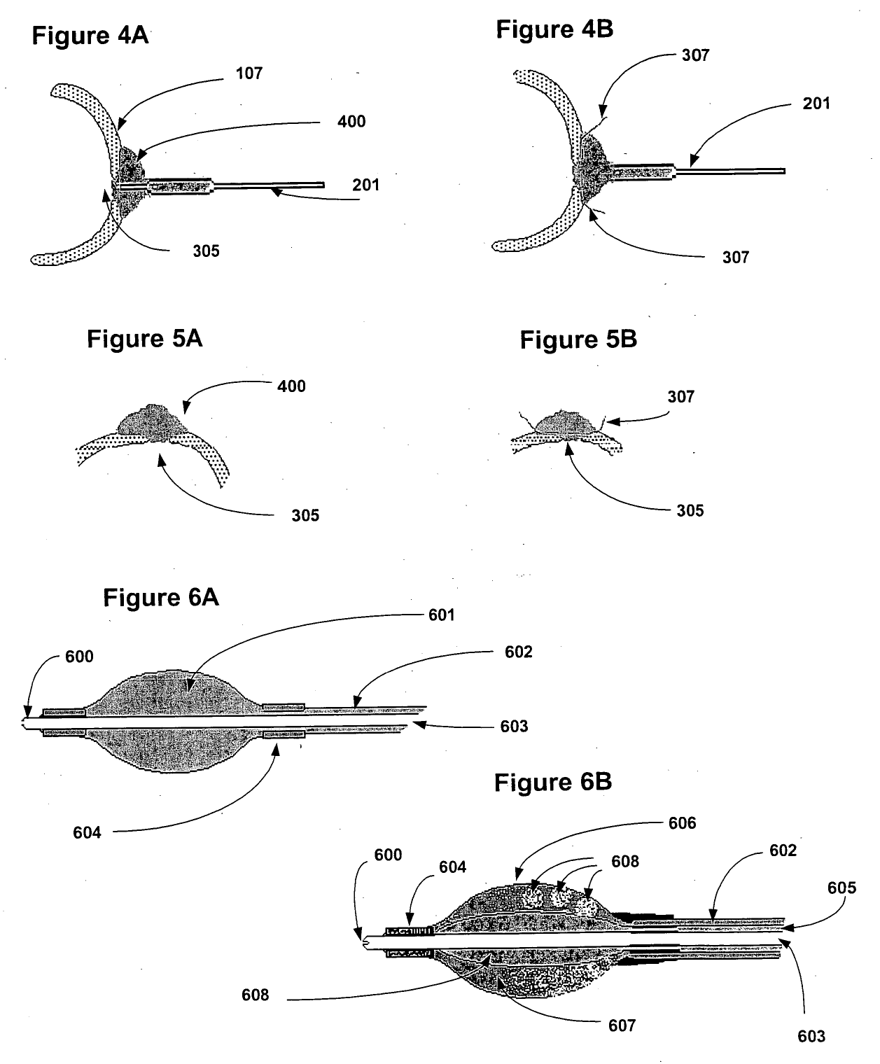

FIG. 4A illustrates the method of transpericardial tissue channel sealing the in greater detail. Tissue sealant 400 can be constantly supplied to the place of sealing by transporting from the syringe 208 via injection line 203. Once a sufficient deposit of tissue sealant is built over puncture the anchoring/sealing balloon 201 is removed. After a few minutes, the released tissue sealant 400 creates a reliable puncture closure by filling the tissue channel 305 and depositing on the outer surface of the pericardium.

Yet another way of closing the transcardial incision shown in FIG. 4B is by application of the surgical suture 307 and purse stringing it around the balloon 201 while the balloon is partially deflated but still in place Once balloon is removed from the incision, the balloon is reinflated with tissue sealant and pressed firmly against the puncture combined tissue approximation can be achieved via tightening sutures up simultaneously with sealant release and deposition as described above.

FIG. 5A illustrates a final result of the puncture closure with tissue sealant 400 abundantly deposited within and over the tissue channel 305. FIG. 5B illustrates a final result of the puncture closure utilizing combined approach with tissue sealant 400 abundantly deposited within and over the tissue channel 305 and the edges of the incision approximated using surgical suture 307.

FIGS. 6 A, B illustrate the structure and functions of the inflatable anchoring and sealing balloon in greater detail. The inflatable anchoring balloon can be inflated by infusion of saline 601 via inflation lumen 602 communicating to and utilized for the securing of the cannula. The central lumen 603 of the balloon is communicating with the source of BES via injection line 204. The distal end of the central lumen 600 has an opening and is in fluid communication with the pericardial space 106. The balloon can be made out of a silicon elastomer such as Silastic and bonded using heat shrink tubing 604 such as PTFE to the shaft of the cannula.

The balloon can also be inflated by infusion of the tissue sealant such as BioGlue produced by CryoLife Inc. Using the tissue sealant enables the inflatable balloon to provide a dual function of anchoring the cannula in place and sealing of the transpericardial incision at the end of the procedure.

Besides securing the cannula in place and prevention of the BES leakage during the injection, the dual function balloon can deliver a tissue sealant directly to the insertion site. The combined anchoring and sealing mechanism demonstrated by FIGS. 3E, F and 4A, B provides a capability for rapid and reliable closure of the transpericardial insertion site.

Alternative embodiment of the combined anchoring and sealing mechanism depicted in FIG. 6B is dual chamber balloon. It utilizes two coaxial, inflatable balloons with multiple orifices or pores 304 on the working length of the outer balloon 606. The central lumen 603 of the balloon is communicating with the source of BES via injection line 204. Prime lumen 605 communicating with the inner balloon 608 and is used to deliver inflation media such as saline 601 transporting from the syringe 207 via injection line 202 for the anchoring during the first (BES injection) phase of the procedure. Secondary lumen 602 is communicating with outer porous balloon 606 and is used for delivery of the tissue sealant 607 by transporting from the syringe 208 via injection line 203 during the closure phase of the procedure. Pores or multiple orifices are located predominantly on the posterior portion of the outer balloon 606 for better deposition over the puncture wound. Both balloons can be constructed using standard catheter building materials such as silicone elastomers and polyurethanes and can be attached to the shaft of the cannula with heat shrink tubing 604 such as PTFE.

The injectable biodegradable viscoelastic substance 304 used to create a hydraulic heart constrainer may be one or more biodegradable biomaterials. It may be chosen from the natural biopolymers and substances such as: lipids, collagen, polysaccharides in the form of proteoglycans and glycosaminoglycans and polyglyconates specifically hyaluronic acid (HA) and its derivatives, cellulose, gelatin, starch, as well as synthetic polymers such as polylactide (PLA), a polyglycolide (PGA), poly(lactide-co-glycolide) (PLGA), polyanhydride, PEG, and/or polyorthoesters. The desirable injectable biodegradable viscoelastic substance may have an array of properties allowing it to produce a therapeutic effect during a desirable therapeutic window and to dissipate afterwards without any toxic product of degradation. The therapeutic window is determine to be between 14 and 60 days. In order to produce desirable therapeutic effect, BES should have a viscosity range of 10000 CST to 15000 CST. The desirable induced interpericardial pressure should be in the range of 12 mmHg to 32 mmHg. That combination of requirements suggests a number of potential candidates from the above described groups of biomaterials. In this preferred embodiment a chosen material is a cross linked collagen gel. Collagen has been used extensively in medicine and in surgery. Collagen is a fibrous protein and constitutes the major protein component of skin, bone, tendon, ligament, cartilage, basement membrane and other forms of connective tissue. Collagen based devices have been used as nerve regeneration tubes, as sutures, haemostatic fiber and sponges, wound dressings, neurosurgical sponges, injectable implants for soft tissue augmentation, pharmaceutical carriers, ophthalmic aqueous-venous shunts, contact lenses and the like. The Injectable collagen products that gained widespread use for subcutaneous, subdermal, and intradermal and periuretheral injections are commercially available and sold under different names such as Zyderm, Zyplast produced by Collagen Corporation and Contigen produced by CR Bard. Typically Injectable collagen material is suspended in media tailored for the specific application, and is packaged in syringes ready for injection through small gauge needles.

The properties of collagen which favor its use as a biomaterial are many. Collagen is biodegradable, and when implanted in the body, is absorbed at a rate that can be controlled by the degree of intra or intermolecular cross-linking imparted to the collagen molecule by chemical or physical treatment. Collagen products can thus be designed such that, on implantation, they will completely be absorbed in a few days or in months. The collagen can also be chemically treated so that it becomes non-absorbable while still retaining its hydrophilic character and its good tissue response.

The main sources of collagen for commercial applications are bovine tendons, calf, steer or pig hide. All are readily available at relatively low cost. Generally, reconstituted collagen products are prepared by purification of native collagen by enzyme treatment and chemical extraction. The purified collagen is then dispersed or dissolved in solution, filtered and retained as such, or is reconstituted into fiber, film or sponge by extrusion or casting techniques which are well known to those skilled in the art. Suitable collagen material for the hydraulic heart constrainer implant may be available from, for example, DEVRO, Integra Life Sciences, Collagen Matrix and Kensey Nash, among others. The present invention preferably employs collagen in acid swollen solution as a starting material. An acidic solution of an atelopeptide form of bovine skin collagen is commercially available from DEVRO Pty. Limited. Typically this material is in a solution or gel form with concentration in the range of about 4-60 mg/mL. The concentration of collagen can be adjusted downwards, if necessary, by simple dilution to achieve desirable viscosity.

The embodiments of the present hydraulic heart constrainer implant may be selectively biodegradable and/or bio-absorbable such that it degrades and/or is absorbed after its predetermined useful lifetime is over. An effective way of controlling the rate of biodegradation of embodiments of the present hydraulic heart constrainer implant is to control and selectively vary the number and nature (e.g., intermolecular and/or intramolecular) of crosslinks in the implant material. Control of the number and nature of such collagen crosslinks may be achieved by chemical and/or physical means. Chemical means include the use of such bifunctional reagents such as aldehyde or cyanamide, for example. Physical means include the application of energy through dehydrothermal processing, exposure to UV light and/or limited radiation, for example. Also, a combination of both the chemical and the physical means of controlling and manipulating crosslinks may be carried out. Aldehydes such as glutaraldehydes, for example, are effective reagents of collagenous biomaterials. The control and manipulation of crosslinks within the collagenous solution or gel of the present hydraulic heart constrainer implant may also be achieved, for example, through a combination of dehydrothermal crosslinking and exposure to cyanamide.

Yet another embodiment of the present hydraulic heart constrainer implant may be Hyaluronic Acid or hyluronan which is a naturally occurring, high viscosity, linear mucopolysaccharide comprised of alternating glucuronic acid and N-acetyl-glucosamine residues that interacts with other proteoglycans to provide stability and elasticity to the extracellular matrix of all tissues. Hyaluronic acid is a clear, viscous fluid, manufactured and commercially available from numerous domestic and foreign vendors and sold under different names such as AmVisc and OrthoVisc produced Anika Therapeutics, just to name a few. It is used for ophthalmic vitreous body implants, viscosurgery, and synovial joint replacements. Purified hyaluronic acid is believed to cause very little tissue reaction once spilled into the soft tissue. Suitable Hyaluronic Acid for the hydraulic heart constrainer implant may be available from, for example from Biomatrix Inc, Anika Therapeutics, Genzyme Corp. Lifecore Biomedical, among others.

There are many ways in which hyluronan can be crosslinked to resist enzymatic biodegradation. An effective way of controlling rate of biodegradation of embodiments of the present hydraulic heart constrainer implant is to utilize a small amount of an aldehydes such as glutaraldehydes or formaldehyde to produce a BES with very desirable properties. Crosslinking can also be achieved with divinyl sulfone and polyvalent cations (ferric, aluminum, etc.) and aziridines (e.g. cross-linker CX-100).

The hydraulic heart constrainer implant may also contain a therapeutic or biologically active agent and combinations thereof such as angiogenesis-promoting factors, vascular endothelial growth factor (VEGF), peptides, oligopeptides, just to mention a few.

The amount of injected injection of biodegradable substance should be sufficient to: (a) distribute substance around in-between the heart and the sack, and b) constraint the heart so that its size is substantially reduced as a result of tension generated by the substance. For an adult human heart, the amount of biodegradable substance to be injected is preferably in a range of 40 ml to 100 ml, but greater or less amounts of the biodegradable substance may achieve the desired therapeutic effect of constraining the heart. The biodegradable substance may constrain the heart by at least partially enveloping the heart mussel circumferentially and squeezing or reducing diameter of the enveloped portion of the heart. The aperture in the pericardium made to inject the biodegradable substance is to be sealed after the injection. The aperture may be sealed by injection of a sealing substance, e.g., a biocompatible glue, a suture or other means which ensures that the aperture will not allow for the leakage of a substantial amount of the biodegradable substance.

In an exemplary, method a therapeutic amount of biodegradable substance is injected into the pericardium in an amount sufficient to envelope a substantial portion (or all) of the heart mussel, the injection aperture in the pericardium is sealed after injection, the heart is constrained (e.g., reduced by 10% of the heart volume) as a treatment for acute MI, and the biodegradable substance dissipates in the body after two weeks to twelve weeks (more or less) and preferably within a period of four to eight weeks.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. A system for injecting a biodegradable pericardial constraint comprising:
a biodegradable viscoelastic substance (BES);
an external injection container for the BES;
a biocompatible sealant;
an external injection container for the sealant;
a cannula having a distal section adapted to be inserted into a pericardial sac of a mammalian heart and having a proximal section connectable to the external injection container, and
wherein BES from the injection container is injected into the pericardial sac through the cannula in an amount sufficient to constrain the heart to achieve a therapeutic effect and the sealant is injected into the pericardial sac through the cannula in an amount sufficient to seal an aperture formed in the pericardial sac after injection of the BES.

2. The system of claim 1 wherein the BES comprises a natural biopolymer.

3. The system of claim 1 wherein the BES is selected from a group consisting of lipids, collagen, polysaccharides and polyglyconates, cellulose, gelatin and starch.

4. The system of claim 1 wherein the BES comprises a crosslinked collagen gel.

5. The system of claim 1 wherein the BES comprises a Hyaluronic Acid.

6. The system of claim 1 wherein the BES is a synthetic polymer.

7. The system of claim 1 wherein the BES is selected from a group consisting of polylactide (PLA), polyglycolide (PGA), poly(lactide-co-glycolide) (PLGA), polyanhydride, PEG and polyorthoesters.

8. The system of claim 1 wherein the BES comprises at least one of angiogenesis-promoting factors, vascular endothelial growth factor (VEGF), peptides, and oligopeptides.

9. The system of claim 1 wherein the BES has a viscosity in a range of 10,000 CST to 15,000 CST.

10. The system of claim 1 wherein the external injector comprises a syringe containing the BES.

11. The system of claim 1 wherein the external injector comprises a power injector applying pressure to the BES during injection into the cannula.

12. The system of claim 1 wherein the distal section of the cannula comprises a balloon which seals and anchors the distal section to the sac.

13. The system of claim 12 wherein the balloon is inflated by infusion of a tissue sealant and wherein the distal section further comprises a perforator to perforate the balloon.

14. A method comprising:
inserting a cannula through a transpericardial incision and into a pericardial space of a heart of a mammalian patient,
connecting a delivery system containing a biodegradable viscoelastic substance to the cannula;
injecting the biodegradable viscoelastic substance injection into the pericardial space, and
sealing the transpericardial incision after injection of the biodegradable viscoelastic substance.

15. The method of claim 14 wherein the injection of the BES comprises injecting a volume of the BES in a range of 40 milliliters (ml) to 80 ml into the space.

16. The method of claim 14 wherein the introduction of the BES comprises power injecting the BES under pressure into the cannula.

17. The method of claim 14 further comprising extracting the cannula, and sealing the transpericardial incision with a suture.

18. The method of claim 17 further comprising sealing the cannula transpericardial incision by injection of a sealing material through the cannula while or after the cannula is withdrawn from the incision.

19. The method of claim 14 wherein the BES comprises a natural biopolymer.

20. The method of claim 14 wherein the BES is selected from a group consisting of lipids, collagen, polysaccharides and polyglyconates, cellulose, gelatin and starch.

21. The method of claim 14 wherein the BES comprises a crosslinked collagen gel.

22. The method of claim 14 wherein the BES comprises a Hyaluronic Acid.

23. The method of claim 14 wherein the BES is a synthetic polymer.

24. The method of claim 14 wherein the BES is selected from a group consisting of polylactide (PLA), polyglycolide (PGA), poly(lactide-co-glycolide) (PLGA), polyanhydride, PEG and polyorthoesters.

25. The method of claim 14 wherein the BES comprises at least one of angiogenesis-promoting factors, vascular endothelial growth factor (VEGF), peptides, and oligopeptides.

26. The method of claim 14 wherein the BES has a viscosity in a range of 10,000 CST to 15,000 CST.

27. The method of claim 14 wherein the external injector comprises a syringe containing the BES.

28. The method of claim 14 wherein the external injector comprises a power injector applying pressure to the BES during injection into the cannula.

29. The method of claim 14 wherein the distal section of the cannula comprises a balloon which seals and anchors the distal section to the sac.

30. The method of claim 29 wherein the balloon is inflated by infusion of a tissue sealant and further comprising perforating the balloon to apply tissue sealant to seal an incision through which the cannula was introduced.

31. The method of claim 14 further comprising dissipating the BES in the sac.

32. The method of claim 14 further comprising dissipating the BES in the sac in a period between 14 to 60 days.

33. The method of claim 14 further comprising monitoring interpericardial pressure and injecting the BES to raise the interpericardial pressure to be in a range of 12 mmHg to 32 mmHg.

34. A treatment system for a biodegradable pericardial constraint comprising:
a cannula placed in a pericardial space of a heart of a mammalian patient;
an external system connectable to the cannula for delivery of a hydraulic heart constrainer in a controlled manner,
a biodegradable viscoelastic substance (BES) to be delivered by the external system through a transpericardial incision to the pericardial space, wherein the BES constrains the heart constrainer when in the pericardial space, and
a sealer applied to the transpericardial incision in the pericardial space.

35. A method to constrain a mammalian heart of a patient comprising:
positioning a cannula through a transpericardial incision and into a pericardial sac of the heart;
introducing a biodegradable viscoelastic substance (BES) though the cannula into the pericardial sac;
extracting the cannula from the pericardial sac after introducing the BES,
sealing the transpericardial incision, and
decomposing the BES into the patient.
